(19) United States
(12) Patent Application Publication
(10) Pub. No.: US 2018/0228825 A1
(43) Pub. Date: Aug. 16, 2018

(54) SULFOLIPIDS AS NEW GLUTAMINYL CYCLASE INHIBITORS

(71) Applicants: Hochschule Anhalt, Kothen (DE); Leibniz-Institut Für Pflanzenbiochemie (IPB), Halle (Saale) (DE)

(72) Inventors: Stephanie Hielscher-Michael, Konnern (DE); Carola Griehl, Halle (Saale) (DE); Hans-Ulrich Demuth, Halle (Saale) (DE); Stephan Schilling, Halle (Saale) (DE); Ludger Wessjohann, Halle (Saale) (DE); Norbert Arnold, Halle (Saale) (DE)

(73) Assignees: Hochschule Anhalt, Kothen (DE); Leibniz-Institut Für Pflanzenbiochemie (IPB), Halle (Saale) (DE)

(21) Appl. No.: 15/761,060

(22) PCT Filed: Sep. 15, 2016
(86) PCT No.: PCT/EP2016/071846

§ 371 Date: Mar. 16, 2018

(30) Foreign Application Priority Data
Sep. 16, 2015 (DE) 10 2015 011 780.7

(51) Int. Cl. A61K 31/7028; A61K 36/02; A61K 45/06; C12P 19/44

(52) U.S. Cl. CPC A61K 31/7028; C12P 19/44; A61K 45/06; A61K 36/02

(57) ABSTRACT

The invention relates to the use of a compound of formula (I), or a composition containing the compound, as glutaminyl cyclase (QC) inhibitor and to methods of preparation.

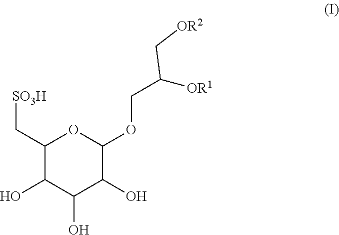

### FIELD OF THE INVENTION

The invention relates to the use of a compound, or a composition containing the compound, as glutaminyl cyclase (QC) inhibitor and to methods of preparation.

### PRIOR ART

The glutaminyl cyclases (QCs) (EC 2.3.2.5) belong to the class of acyltransferases (EC 2.3.2). QC catalyses the intra-molecular cyclisation of N-terminal L-glutamine residues of peptides and proteins to form pyroglutamic acid (pGlu), with ammonia being released, and the intramolecular cyclisation of N-terminal glutamate to form pyroglutamic acid, with water being released. QC plays a part in a large number of physiological processes and it has been shown that QC is involved pathophysiologically in a variety of diseases. Accordingly, QC is a pharmacologically interesting target, because the inhibition of QC is regarded as a highly promising option for the treatment of such QC-associated disorders/conditions.

A variety of QC inhibitors are already known. For example, thiourea derivatives and imidazole-propyl-thioamides have been identified as effective QC inhibitors by Buchholz et al. (J Med Chem (52), 7069-7080; 2006). Further QC inhibitors are described, for example, in WO 2010/026212, WO 2011/029920; WO 2011/101433; WO 2011/107530; and WO 2011/110613. QC inhibitors from natural sources have not been known hitherto.

Sulfolipids are known as biologically active natural substances. The bioactivities previously described for sulfolipids include: immunosuppressive effects, antiviral effects (e.g. against HIV), antineoplastic effects, an inhibiting effect on the enzymatic activities of the DNA polymerases pol α and pol β, of α-glucosidase and of caspase, anti-inflammatory activities (e.g. an effect in the case of inflammatory skin disorders or disorders such as, especially, psoriasis) and anti-proliferative effects on various human cancer cell lines, a prophylactic effect against a Mycobacterium tuberculosum infection, and an antiprotozoal activity.

A common problem of inhibitors is their side-effects, such as, for example, cytotoxicity. The increasing occurrence of incompatibilities with respect to certain active ingredients or molecular constituents also represents a serious problem. Accordingly there is a great need for inhibitors without cytotoxic effect.

The problem of the present invention was the provision of new QC inhibitors that originate from natural sources, have no cytotoxic effects and are usually well tolerated.

### DESCRIPTION OF THE INVENTION

The invention relates to the use of a compound, or a composition containing the compound, as glutaminyl cyclase (QC) inhibitor and to methods of preparation.

For that purpose the present invention provides a compound of formula (I):

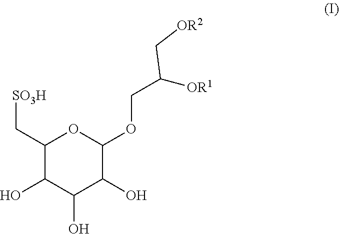

or a salt thereof, for use as glutaminyl cyclase (QC) inhibitor, wherein

R¹ is a hydrogen atom, —C(═O)C₁-C₂₆alkyl or —C(═O)C₂-C₂₆-alkenyl; and

R² is a hydrogen atom, —C(═O)C₁-C₂₆alkyl or —C(═O)C₂-C₂₆-alkenyl.

The term “C₁-C₂₆alkyl”, as used herein, denotes an alkyl group and signifies a saturated, straight-chain or branched, optionally substituted hydrocarbon group having from 1 to 26 carbon atoms. Representative “C₁-C₂₆alkyl” radicals are e.g. the methyl, ethyl, propyl, butyl, pentyl (or valeryl), hexyl (or caproic), heptyl (or oenanthyl), octyl (or caprylic), nonyl (or pelargonyl), decyl (or capric), undecyl; dodecyl (or lauryl), tridecyl, tetradecyl (or myristyl), pentadecyl, hexadecyl (or cetyl), heptadecyl, octadecyl (or stearyl); nonadecyl, eicosyl, heneicosyl, doeicosyl, tricosyl, tetra-cosyl, pentacosyl, hexacosyl, isopropyl, sec-butyl, isobutyl, tert-butyl, isopentyl, 2-methylbutyl and 2-methylhexyl groups.

The term “—C₂-C₂₆alkenyl”, as used herein, denotes an alkenyl group and signifies an at least partially unsaturated, straight-chain or branched, optionally substituted hydrocarbon group having from 2 to 26 carbons atoms. Representative “—C₂-C₂₆alkenyl” radicals are e.g. vinyl, allyl, 1-butenyl, 2-butenyl, -isobutylenyl, 1-pentenyl, 2-pentenyl, 3-methyl-1-butenyl, 2-methyl-2-butenyl, 2,3-dimethyl-2-butenyl-ethenyl, hex-2-enyl, 9-hexadecenyl (or palmitoleyl), 9-octadecenyl (oleyl), 11-octadecenyl, 9,12-octadecadienyl (or linoleyl), 6,9,12-octadecatrienyl (or linolenyl), 5,8,11,14-eicosatetraenyl or 5,8,11,14,17-eicosapentaenyl. The alkenyl groups contain one or more double bond(s), preferably 1, 2, 3, 4 or 5 double bond(s).

The expression “optionally substituted”, as used herein, denotes groups in which one or more hydrogen atom(s) has/have been replaced by one or more fluorine, chlorine, bromine or iodine atom(s), OH, ═O, SH, ═S, NH₂, ═NH, NO₂, —C(O)R′, —OC(O)R′, —C(O)OR′, —C(O)NH₂, —C(O)NHR′, —C(O)N(R′)₂, —NHC(O)R′, —S(O)₂R′, —S(O)R′, —N₃, —NH(R′), —N(R′)₂ or —CN, preferably by one or more fluorine or chlorine atom(s), OH, C(O)R′, —OC(O)R′ or —C(O)OR′, more preferably by one or more fluorine or chlorine atom(s), or OH, wherein R′ is in each case selected independently from unsubstituted C₁-C₃alkyl.

The compound of formula (I) can, depending upon its structure, exist in stereoisomeric forms (enantiomers, diastereoisomers). The invention therefore comprises the enantiomers or diastereoisomers and also mixtures thereof. The stereoisomerically uniform constituents can be isolated from such mixtures of enantiomers and/or diastereoisomers in known manner, e.g. by means of column chromatography.

If the compound of formula (I) can occur in tautomeric form, the present invention comprises all tautomeric forms.

As salts, within the scope of the present invention preference is given to physiologically acceptable salts of the compound of formula (I). Also included, however, are salts that are themselves unsuitable for physiological applications but can be used, for example, as a research tool, in cosmetic applications, or for the isolation or purification of the compound or other molecules.

Physiologically acceptable salts of the compound of formula (I) can also be acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid. Furthermore, physiologically acceptable salts of the compound of formula (I) can also include salts of customary bases, such as, for example, alkali metal salts (e.g. sodium and potassium salts); alkaline earth metal salts (e.g. calcium and magnesium salts); and ammonium salts, derived from ammonia or organic amines having from 1 to 16 carbon atoms, such as, for example, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Preference is given to a compound of formula (I) or a salt thereof, for use as QC inhibitor, wherein
R¹ is a hydrogen atom, —C(═O)C₁₃-C₂₀alkyl or —C(═O)C₁₃-C₂₁-alkenyl; and
R² is a hydrogen atom, —C(═O)C₁₃-C₂₁alkyl or —C(═O)C₁₃-C₂₁-alkenyl.

Preference is given also to a compound of formula (I) or a salt thereof, for use as QC inhibitor, wherein
R¹ is a hydrogen atom, a palmitoyl, palmitoleoyl, oleoyl, linoleoyl, linolenoyl, eicosatetraenoyl or an eicosapentaenoyl group; and
R² is a hydrogen atom, a palmitoyl, palmitoleoyl, oleoyl, linoleoyl, linolenoyl, eicosatetraenoyl or an eicosapentaenoyl group.

According to the invention preferably the compound of formula (I) or a salt thereof, for use as QC inhibitor, can be selected from the group: 1,2-di-O-palmitoyl-3-O-(6′-deoxy-6′-sulfo-D-glycopyranosyl, 1-O-palmitoyl-2-O-linolenyl-3-O-(6′-deoxy-6′-sulfo-D-glycopyranosyl, 1-O-linolyl-2-O-palmitoyl-3-O-(6′-deoxy-6′-sulfo-D-glycopyranosyl, 1-O-palmitoyl-3-O-(6′-sulfo-α-quinovopyranosyl)-glycerol and 1-O-(6-deoxy-6-sulfoglucopyranosyl)-glycerol.

The invention relates also to the use of the compound according to formula (I) or a salt thereof, as QC inhibitor, wherein R¹ is a hydrogen atom, —C(═O)C₁-C₂₆alkyl; or —C(═O)C₂-C₂₆alkenyl; and R² is a hydrogen atom, —C(═O)C₁-C₂₆alkyl; or —C(═O)C₂-C₂₆-alkenyl. There can preferably be used as QC inhibitor a compound of formula (I) or a salt thereof, wherein R¹ is a hydrogen atom, —C(═O)C₁₃-C₂₀alkyl, or —C(═O)C₁₃-C₂₁alkenyl; especially a hydrogen atom, a palmitoyl, palmitoleoyl, oleoyl, linoleoyl, linolenoyl, eicosatetraenoyl, or an eicosapentaenoyl group; and R² is a hydrogen atom, —C(═O)C₁₃-C₂₀alkyl, or —C(═O)C₁₃-C₂₁alkenyl; especially a hydrogen atom, a palmitoyl, palmitoleoyl, oleoyl, linoleoyl, linolenoyl, eicosatetraenoyl, or an eicosapentaenoyl group. In particular, 1,2-di-O-palmitoyl-3-O-(6′-deoxy-6′-sulfo-D-glycopyranosyl, 1-O-palmitoyl-2-O-linolenyl-3-O-(6′-deoxy-6′-sulfo-D-glycopyranosyl, 1-O-linolyl-2-O-palmitoyl-3-O-(6′-deoxy-6′-sulfo-D-glycopyranosyl, 1-O-palmitoyl-3-O-(6′-sulfo-α-quinovopyranosyl)-glycerol, or 1-O-(6-deoxy-6-sulfoglucopyranosyl)-glycerol can be used as QC inhibitor. The use can take place in vitro or in vivo. A use in vivo relates, for example, to the compound according to formula (I) or a salt thereof, for use in the prophylaxis or treatment of a QC-associated illness. When used in vivo, however, methods for the surgical or therapeutic treatment of the human or animal body and diagnostic procedures performed on the human or animal body are excluded. However, the use of the compound according to formula (I) or a salt thereof, for the preparation of a medicament for use in the prophylaxis or treatment of a QC-associated illness is included.

The present invention relates also to a composition, comprising one of the afore-mentioned compounds according to formula (I) or a salt thereof, for use as QC inhibitor. Preferably the composition comprising a compound according to formula (I) or a salt thereof can be an extract from a microalga. For obtaining the extract there are suitable, for example, microalgae of the species: Scenedesmus sp., e.g. Scenedesmus producto-capitatus, Scenedesmus rubescens, Scenedesmus acuminatus, Scenedesmus vacuolatus and Scenedesmus pectinatus and also Tetradesmus wisconsinensis; Eustigmatos sp., e.g. Eustigmatos magnus, Cyclotella sp., Odontella sp., Synechocystis sp., Spirulina sp., e.g. Spirulina maxima and Spirulina platensis; Prochlorococcus sp., Synechococcus sp., Trichodesmium erythraeum, Arthrospira platensis, Scytonema hofmanni, Nostoc punctiforme, Microcystis aeruguinosa, Stephanodiscus sp., Skeletonema costatum, Phaeodactylum tricornutum, Heterosigma carterae, Nannochloropsis sp., e.g. Nannochloropsis oculata, Porphyridium sp., e.g. Porphyridium cruentum and Porphyridium purpureum; Chilomonas paramecium, Bumilleriopsis filiformis, Emiliana huxleyi, Chlorella sp., e.g. Chlorella vulgaris, Chlorella sorokiniana, Chlorella variegate, Chlorella kessleri, Chlorella minutissima and Muriella zofingiensis; Chloroidium saccharophilum, Parietochloris incisa, Prototheca portoricensis, Dictyochloris fragrans, Chlamydomonas reinhardtii, Dunaliella parva, Tetraselmis chuii and Deasonia sp., e.g. Deasonia punctata and Deasonia granata; and especially preferred examples of the microalgae extracts comprise extracts which can be obtained from a species selected from Scenedesmus acuminatus, Scenedesmus pectinatus, Chlorella vulgaris and Spirulina platensis.

The composition or the microalgae extract for use as QC inhibitor can also contain a solvent. The solvent can be selected, for example, from: methanol, ethanol, isopropanol, water, chloroform, ethyl acetate, dichloromethane, acetone, diethyl ether, hexane, and mixtures thereof; preferably methanol.

The composition or the microalgae extract for use as QC inhibitor can also comprise two, three or more than three compounds according to formula (I) or salts thereof.

The composition or the microalgae extract for use as QC inhibitor can also comprise a further therapeutic agent selected from the group comprising acetylcholinesterase inhibitors (e.g. Aricept®, Exelon® and Reminyl®), NMDA-receptor antagonists (e.g. memantine), neuroprotectors, anti-Parkinson medicaments, antidepressives, anxiolytic medicaments, antipsychotic medicaments, medicaments against multiple sclerosis, ACE inhibitors (e.g. Enap, enalapril, Capoten, Renitec, Prestarium (Berlipril, Diroton, Capoten, Quadropril, Monopril, Renitec, Prestarium, Noliprel-Forte, Enap-N), diuretics (e.g. furosemide, Verospiron, hypothiazide, Arifon Retard, indapamide, hypothiazide, Diuver, indap, indapamide), p-receptor blockers, nitrates, cardiac glycosides, calcium antagonists (e.g. (Normodipine, Cordaflex, Amlovas, amlodipine, Amlovas, Amlotop, cardilopine, Cordaflex, lipid reducers (e.g. (Vasilip, Liprimar, Liptonorm, Simvahexal, Simvastol, Simvacard, Simgal, tulip), aggregation inhibitors (e.g. (acetylsalicylic acid, CardiASK, Cardiomagnyl, Thrombo ASS), antihypoxaemics, coagulation inhibitors (e.g. thrombo-modulin, warfarin), cytostatics and antibiotics.

The composition or the microalgae extract for use as QC inhibitor can also contain a carrier, preferably a pharmacologically inert, inorganic or organic excipient, such as e.g. mannitol, lactose, sucrose, glucose, gelatin, malt, silica gel, starch or derivatives thereof, talcum, stearic acid or salts thereof, dried skimmed milk, vegetable oils, petroleum, animal or synthetic oils, waxes, fats, polyols. The composition or microalgae extract for use as QC inhibitor can also contain additives for preserving, stabilisation, emulsifiers, sweeteners, flavourings, salts for altering the osmotic pressure, buffers, coating ingredients and antioxidants.

Furthermore, the invention relates also to a method of preparing a compound of formula (I) in accordance with the above statements, or a salt thereof, comprising:

(a) culturing at least one microalga;

(b) obtaining the algal biomass and disrupting the algal cells;

(c) extracting the disrupted algal cells from step (b) with methanol, comprising suspending the disrupted algal cells from step (b) in methanol, stirring the suspension, and separating the liquid methanol extract phase from the cell mass;

(d1) isolating a compound of formula (I), or a salt thereof, from the extraction product obtained in step (c).

In the method of preparing a compound of formula (I) there can be used as microalga any microalga that produces compounds of formula (I) as secondary metabolite or secondary metabolites, or a mixture of such microalgae, for example a microalga of the phyla Bacillariophyta, Chlorophyta, Ochrophyta and Cyanobacteria, and preferably of the species: Scenedesmus sp., e.g. Scenedesmus producto-capitatus, Scenedesmus rubescens, Scenedesmus acuminatus, Scenedesmus vacuolatus and Scenedesmus pectinatus and also Tetradesmus wisconsinensis; Eustigmatos sp., e.g. Eustigmatos magnus, Cyclotella sp., Odontella sp., Synechocystis sp., Spirulina sp., e.g. Spirulina maxima and Spirulina platensis; Prochlorococcus sp., Synechococcus sp., Trichodesmium erythraeum, Arthrospira platensis, Scytonema hofmanni, Nostoc punctiforme, Microcystis aeruginosa, Stephanodiscus sp., Skeletonema costatum, Phaeodactylum tricornutum, Heterosigma carterae, Nannochloropsis sp., e.g. Nannochloropsis oculata, Porphyridium sp., e.g. Porphyridium cruentum and Porphyridium purpureum; Chilomonas paramecium, Bumilleriopsis filiformis, Emiliana huxleyi, Chlorella sp., e.g. Chlorella vulgaris, Chlorella sorokiniana, Chlorella variegate, Chlorella kessleri, Chlorella minutissima and Muriella zofingiensis; Chloroidium saccharophilum, Parietochloris incisa, Prototheca portoricensis, Dictyochloris fragrans, Chlamydomonas reinhardtii, Dunaliella parva, Tetraselmis chuii and Deasonia sp., e.g. Deasonia punctata and Deasonia granata; and especially preferably Scenedesmus acuminatus, Scenedesmus pectinatus, Chlorella vulgaris and Spirulina platensis.

The culturing of the microalga(e) according to step (a) can be carried out under known conditions, for example in a photobioreactor, pH 5-8 (preferably pH 7), and a temperature of 10-45° C., preferably 20-40° C., more preferably 23-35° C., and especially preferably 25-31° C. Suitable as medium for culturing the microalga are known media, such as, for example, Setlik medium (KNO₃ (2020.00 mg/L), KH₂PO₄ (340.00 mg/L), MgSO₄*7 H₂O (990.00 mg/L), Fe-EDTA (18.50 mg/L), Ca(NO₃)₂*4 H₂O (10.00 mg/L), H₃BO₃ (3.09 mg/L), MnSO₄*4 H₂O (1.20 mg/L), CoSO₄ (1.40 mg/L), CuSO₄*5 H₂O (1.24 mg/L), ZnSO₄ (1.43 mg/L), (NH₄)₆Mo₇O₂₄*4 H₂O (1.84 mg/L)).

Obtaining the algal biomass in step (b) can be effected by one of the numerous known physical or chemical methods for the separation of biomass from a culture medium, for example by centrifuging off the medium for the culturing and/or lyophilisation. The disruption of the algal cells in step (b) can be carried out in a suitable way using one of the numerous known physical or chemical methods for cell disruption of microorganisms, as described e.g. in Ullmann's Encyclopedia of Industrial Chemistry, 2003, Volume 5, Biochemical Separations, Table 3. One possible method is, for example, grinding, e.g. with a mortar and pestle using sea sand (ratio algal biomass: sea sand 1:2) and solvent (e.g. water). The cell disruption can be effected at any desired suitable temperature, e.g. room temperature.

For the extraction of the disrupted algal cells with methanol in accordance with step (c), preferably solid-liquid extraction is used, the extraction being effected with stirring and 100 mL of methanol/1 g of the disrupted algal cells being used in each case. The separation of the liquid methanol extract phase from the biomass can be effected by means of centrifugation, e.g. 1000-5000 rpm, preferably 1500-3000 rpm, more preferably 2000-2500 rpm, and especially at about 2000 rpm. Preferably the biomass can in each case be extracted three times with methanol. The liquid methanol extract phases of the extractions of the biomass can be combined. The combined liquid methanol extract phases can be concentrated to dryness in vacuo, e.g. with the aid of a vacuum rotary evaporator.

The isolation, in accordance with step (d1), of a compound of formula (I) from the extraction product obtained in step (c) can be effected by one of the numerous known physical or chemical methods for the isolation of compounds from an extract, for example by solid phase extraction (SPE). Numerous sorbents are known for the SPE, for example an aminopropyl-modified silica gel phase can be used. As solvent for elution there can be used suitable solvents known for SPE, such as, for example, methanol, ethanol, isopropanol, water, chloroform, ethyl acetate, dichloromethane, acetone, diethyl ether, hexane, and mixtures thereof; preferably methanol. For removal of non-lipid impurities, the product of the SPE can be purified further, for example by means of chromatography or other known purification methods; preferably the product of the SPE can be further purified in accordance with the method of Folch et al. (J. Biol. Chem., 226, 497-509 (1957)). The invention relates also to a method of preparing an extract from a microalga, which extract contains a compound of formula (I) in accordance with the above statements, or a salt thereof, wherein the method comprises:

(a) culturing the microalga;

(b) obtaining the algal biomass and disrupting the algal cells;

(c) extracting the disrupted algal cells from step (b) with methanol, comprising suspending the disrupted algal cells from step (b) in methanol, stirring the suspension, and separating the liquid methanol extract phase from the cell mass;

(d2) removing chlorophyll from the extraction product obtained in step (c).

Steps (a), (b) and (c) of the method of preparing an extract from a microalga can be carried out analogously to steps (a), (b) and (c) of the above-described method of preparing a compound of formula (I).

The removal of chlorophyll from the extraction product obtained can be carried out using a known method, for example by ion exchange chromatography, e.g. using commercially available columns for exchange of cations. As solvents for the ion exchange chromatography it is possible to use known suitable solvents, for example methanol. For further purification of the extract, solid phase extraction can be carried out. Numerous sorbents are known for solid phase extraction, for example an octadecyl-modified silica gel phase can be used. As solvents for elution it is possible to use suitable solvents known for solid phase extraction, preferably methanol.

### BRIEF DESCRIPTION OF THE FIGURES

FIG. 1: Diagram illustrating the method according to the invention for preparing a compound of formula (I) having QC-inhibiting effect or for preparing an extract from a microalga, which extract contains at least one compound of formula (I) having QC-inhibiting effect.

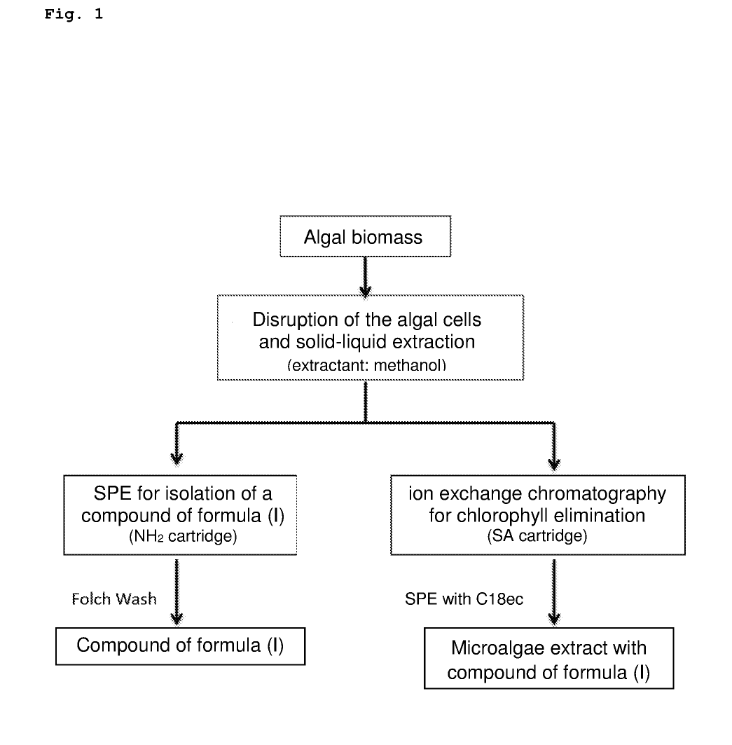

FIG. 2: QC-Inhibiting effect of compositions according to the invention. Concentration-effect graph of the activity of extracts from Eustigmatos magnus, Tetradesmus wisconsinensis, Scenedesmus pectinatus, Scenedesmus acuminatus, Scenedesmus rubescens and Scenedesmus producto-capitatus. As can be seen in FIG. 2, the extracts obtained from microalgae in the exponential growth phase exhibit stronger inhibition of QC than do the extracts obtained from microalgae in the stationary growth phase. It can also be seen from FIG. 2 that the QC inhibition is specific and concentration-dependent.

FIG. 3: Base-peak chromatogram and MS¹ mass spectrum of the compounds according to formula (I) having QC-inhibiting effect isolated using the method according to the invention. Those compounds are also contained in the compositions and microalgae extracts according to the invention.

The MS¹ mass spectrum shows for the retention time of 6.9 min a molecular ion [M-H]⁻ at m/z 555, which can be assigned to a SQMG. The peak at the retention time of 9.9-10 min shows the molecular ions [M-H]⁻ at m/z 794 and 815 in the MS¹ mass spectrum. This is therefore the peak of the SQDG. There are accordingly present as main compounds the sulfolipids 1,2-di-O-palmitoyl-3-O-(6′-deoxy-6′-sulfo-D-glycopyranosyl) with [M-H]⁻ at m/z 793 and also 1-O-palmitoyl-2-O-linolenyl-3-O-(6′-deoxy-6′-sulfo-D-glycopyranosyl) with [M-H]⁻ at m/z 815. Furthermore, the SQMG 1-O-palmitoyl-3-O-(6′-sulfo-α-quinovopyranosyl)-glycerol was detected as secondary compound.

The present invention is explained below with reference to examples.

### EXAMPLES

Obtaining the Algal Biomass

To obtain sufficient biomass, microalgae of the species Eustigmatos magnus, Tetradesmus wisconsinensis, Scenedesmus pectinatus, Scenedesmus acuminatus, Scenedesmus rubescens and Scenedesmus producto-capitatus were cultured in a tubular photobioreactor PBR 100 GS/PL from the company IGV GmbH having a reactor volume of 100 L. The pump speed, the temperature and the pH value were controlled by means of a biostat control unit from the Braun company, the following parameters being set: temperature 28° C.+/−3° C.; pH=7, pump speed 1750 rpm. The reactor system has a flow cell in which both a temperature probe and a pH probe as well as a probe for measuring the optical density (OD) and a pO₂ probe have been integrated. An inline measurement of the following parameters was carried out: temperature, pH value, OD and pump speed. These were recorded over the entire culturing period by means of the MFSC/win software. The cultures in 100 L PBR were likewise carried out at a temperature of 28° C.+/−3° C. Culturing was carried out with a light intensity of 60 μmol/m²*s at OD<20 and a light intensity of 150 pmol/m²*s of the light module at OD>20. The culturing was carried out in a modified fed-batch process.

Setlik medium (Tab. 1) was used for culturing the microalgae.

TABLE 1
Composition of the Setlik medium

| Nutrient salt | Amount used [mg/L] |
|---|---|
| KNO₃ | 2020.00 |
| KH₂PO₄ | 340.00 |
| MgSO₄ * 7 H₂O | 990.00 |
| Fe-EDTA | 18.50 |
| Ca(NO₃)₂ * 4 H₂O | 10.00 |
| H₃BO₃ | 3.09 |
| MnSO₄ * 4 H₂O | 1.20 |
| CoSO₄ | 1.40 |
| CuSO₄ * 5 H₂O | 1.24 |
| ZnSO₄ | 1.43 |
| (NH₄) ₆Mo₇O₂₄ * 4 H₂O | 1.84 |

The nutrient salts were dissolved in 1 L of distilled water and autoclaved for 20 min at 121° C.

The biomasses of the exponential and stationary growth phases of the mentioned microalgae were investigated.

Extracting the Algal Biomass—Methanol Extract

The lyophilised algal biomasses of the exponential and stationary growth phases of each microalga investigated was disrupted with sea sand in a ratio of 1:2 with a mortar and pestle using solvent. The cell disruption was carried out for 10 min at RT.

For extraction, a one-step extraction method based on solid-liquid extraction was used. In that procedure, after cell disruption in each case 10 g of algal biomass was extracted with the solvent methanol. The extraction was carried out in three steps in each case with 100 mL of solvent/1 g of biomass, with stirring on the magnetic stirrer for 1 h at RT. The separation of the liquid extract phase from the biomass was effected by means of centrifugation at 2000 rpm and RT, so that the biomass could be extracted analogously in the second and third extraction steps. The extracts from the three extraction steps were combined and concentrated to absolute dryness by means of a vacuum rotary evaporator from the Büchi company.

Chlorophyll Elimination

The chlorophyll colouring was eliminated from the methanol extracts using CHROMABOND® SA (SCX) from the company Macherey & Nagel. The SA cartridges were coated with 0.5 g of Na₂SO₄ and conditioned with 1 volume unit of dichloromethane/acetone (3:1, v/v). Then the methanol extract was applied in each case. The chlorophyll-free eluate was collected in glass sample containers.

Obtaining Microalgae Extract with Compound of Formula (I)

The chlorophyll-free extracts were then purified over a Chromabond® C18ec cartridge (500 mg/3 mL and 1000 mg/6 mL, Macherey&Nagel). Elution was effected with 100% MeOH.

QC-Inhibiting Effect of the Microalgae Extracts

The chlorophyll-free microalgae extracts purified over Chromabond® C18ec cartridges were investigated for their QC-inhibiting activity. For that purpose, known methods of detecting QC catalysis can be used. For example, it is possible to use the continuous test procedure developed by Schilling et al. (Anal. Biochem. 303, 49-56 (2002); Biol. Chem. 384, 1583-1592 (2003)), which is based on the conversion of the free ammonia by cyclisation of the N-terminal glutamine residues (loc. cit. 2003) and, on the other hand, reaction of the pyroglutamate peptide formed with pyroglutaminylamino-peptidase (pGAP) (loc. cit. 2002), see reaction scheme 1 below. In that procedure, fluorogenic groups are split off by the auxiliary enzyme pGAP, resulting in an increase in fluorescence.

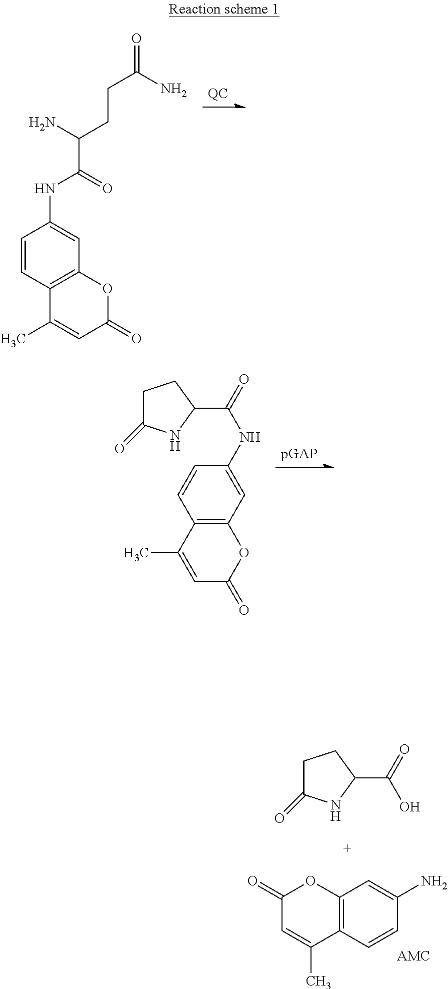

The QC-inhibiting activity of the chlorophyll-free microalgae extracts according to the invention purified over Chromabond® C18ec cartridges was determined in two concentrations (2 mg/mL and 0.2 mg/mL) using the above test procedure of Schilling et al. The test procedure was carried out in triplicate in transparent 96-well microtitre plates (NUNC, Costar Corning Incorporated, Acton, Mass., USA). The test batches had a total volume of 250 μL per well. These were composed of 100 μL 0.25 mM substrate, 50 μL 0.2 mg/mL sample and 25 μL auxiliary enzyme pGAP. Since the QC activity is pH-dependent, 50 μL of Tris buffer (0.1 M; pH 8) were added to each test batch. The start of the reaction was effected after 10 minutes' incubation at 30° C. by addition of 25 μL of QC. The measurements or the increase in free AMC was measured continuously over a period of 12 min (˜27 cycles) at a temperature of 30° C. using a FluoStar Multiplate Reader from the company BMG-Labtech.

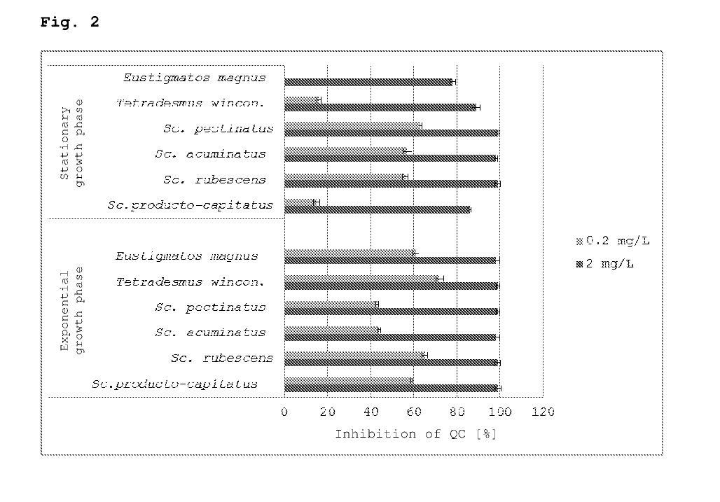

The results of the measurement are shown in FIG. 2. As can be seen from FIG. 2, the microalgae extracts according to the invention inhibit the activity of QC specifically and concentration-dependently.

Identifying the QC Inhibitors in the Microalgae Extracts

For identification of the QC inhibitors, the microalgae extracts according to the invention were analysed by mass spectrometry.

The mass-spectrometric analyses were carried out by means of an API-150EX mass spectrometer having a “Turbolon Spray” ion source from the company Applied Biosystems. The sample injection (10 μL) was effected by means of direct inlet into a continuous flow of a MeOH/H₂O mixture (6:4; v/v) at a flow speed of 400 μL/min.

Furthermore, the microalgae extracts according to the invention were investigated by means of UPLC-ESI-ion trap MSⁿ on an Acquity UPLC system from the Waters company using an RP18 column (Acquity UPLC HSS T3 1.8 μm, 1×100 mm, Waters). The samples were analysed with negative ionisation by means of electrospray ionisation (ESI) at a spray voltage of 4 kV, a capillary temperature of 275° C. and a capillary voltage of 27 V using nitrogen as protective gas (flow rate 35-40 arb. units).

The mass-spectrometric analyses of the extracts investigated indicated that the compounds of formula (I) are responsible for the QC-inhibiting effect. The compounds identified by mass spectrometry were the SQDGs 1,2-di-O-palmitoyl-3-O-(6′-deoxy-6′-sulfo-D-glycopyranosyl with [M-H]⁻ at m/z 793, 1-O-palmitoyl-2-O-linolenyl-3-O-(6′-deoxy-6′-sulfo-D-glycopyranosyl with [M-H]⁻ at m/z 815 and 1-O-linolyl-2-O-palmitoyl-3-O-(6′-deoxy-6′-sulfo-D-glycopyranosyl with [M-H]⁻ at m/z 817 and also the SQMG 1-O-palmitoyl-3-O-(6′-sulfo-α-quinovopyranosyl)-glycerol with [M-H]⁻ at m/z 555 and 1-O-(6-deoxy-6-sulfoglucopyranosyl)-glycerol with [M-H]⁻ at m/z 317.

Obtaining Compounds of Formula (I)

To obtain the QC-inhibiting compounds from the methanol extracts obtained from the algal biomass in the above-described extraction, solid phase extraction with subsequent Folch wash was used.

For the solid phase extraction of the methanol extracts, aminopropyl-modified silica gel cartridges (NH₂ cartridges) from the company Macherey&Nagel (3 mL/500 mg and 6 mL/1000 mg) were used. The conditioning of the NH₂ cartridges was carried out in several steps using 2 mL of MeOH, 2 mL of dist. H₂O, 4 mL of 0.1M HCl (for 1 h on the column), 2 mL of dist. H₂O, 2 mL of MeOH, 2 mL of DCM/isopropanol/MeOH (15:30:50 v/v/v). For each NH₂ cartridge, a sample of 20 mg of methanolic extract, dissolved in DCM/MeOH (1:1; v/v), was applied. When 6 mL/1000 mg NH₂ cartridges were used, the volumes of the solvents used and of the sample applied were doubled.

The elution was carried out in 2 steps. In step 1, first of all the uncharged substance was eluted with 9 ml of DCM/isopropanol/MeOH 15:30:50 (v/v/v). In step 2, the compounds according to formula (I) were eluted with 5 mL of DCM/ACN/isoprop/MeOH/0.1M NH₄Ac (10:10:10:50:15; v/v/v/v/v); fraction 2.

Fraction 2 was freed of the solvent by means of a vacuum rotary evaporator and then redissolved with the addition of DCM/MeOH/0.1 NaAc buffer pH=4.0 (8:4:3, v/v/v) by vigorous shaking for several minutes. After incubation overnight at 4° C., the compounds according to formula (I) became concentrated in the lower, lipophilic phase. The lipophilic phase containing the compounds according to formula (I) was removed using a Pasteur pipette, transferred to a round-bottomed flask and concentrated to dryness by means of a vacuum rotary evaporator.

The yields of the compounds according to formula (I) varied between 3 and 26% in dependence upon the biomass used (algal species and growth phase). For example, from 25 mg of the methanolic extract from Scenedesmus acuminatus (stationary phase) it was possible to obtain compounds according to formula (I) in a yield of 26%, that is to say 6.5 mg were isolated.

Identifying the Compounds Obtained

For identification, the isolated compounds were analysed by mass spectrometry.

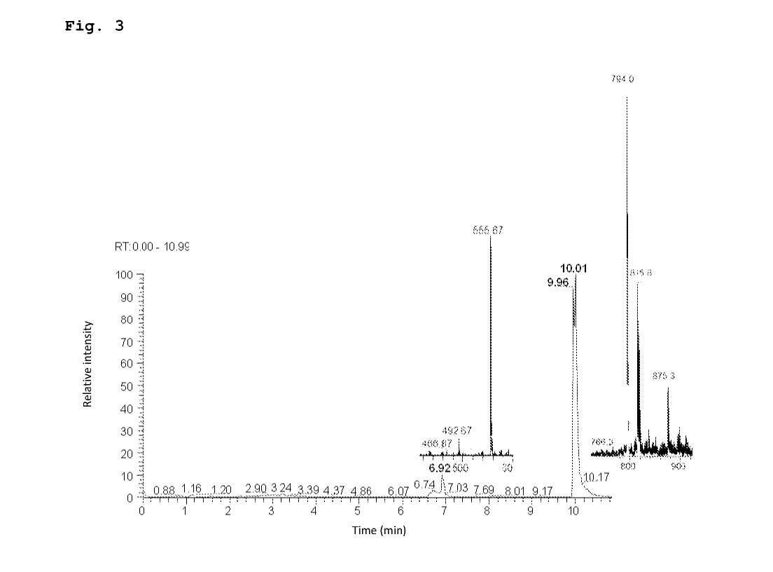

The mass-spectrometric investigations of the isolated compounds were carried out as in the case of the microalgae extracts according to the invention by means of ESI-API-MS and UPLC-MS in negative ion mode. The base-peak chromatogram and the associated MS¹ spectrum is shown in FIG. 3 with the peaks characteristic of compounds according to formula (I) at 9.9-10.0 min and at 6.9 min.

The isolated compounds are compounds according to formula (I), namely the SQDG sulfolipids 1,2-di-O-palmitoyl-3-O-(6′-deoxy-6′-sulfo-D-glycopyranosyl) with [M-H]⁻ at m/z 793 and 1-O-palmitoyl-2-O-linolenyl-3-O-(6′-deoxy-6′-sulfo-D-glycopyranosyl) with [M-H]⁻ at m/z 815, and also the SQMG sulfolipid 1-O-palmitoyl-3-O-(6′-sulfo-α-quinovopyranosyl)-glycerol with [M-H]⁻ at m/z 555.

QC-Inhibiting Effect

In order to confirm that the sulfolipids isolated from the microalga Scenedesmus acuminatus inhibit QC, they were investigated in two concentrations (0.25 mg/mL and 0.025 mg/mL i.a.) using the above-described test procedure of Schilling et al. It was found that sulfolipids isolated from the microalga inhibit QC at 81% (0.25 mg/mL) and 76% (0.025 mg/mL).

As positive control for the QC-inhibiting effect of the compounds according to formula (I) identified as activity-relevant, a SQDG standard from the company Lipid Products in the concentrations 0.25 mg/mL and 0.025 mg/mL i.a. was investigated in the test procedure of Schilling et al. The SQDG standard inhibited the QC by 77% (0.25 mg/mL) and 76% (0.025 mg/mL). The IC₅₀ value determined for the SQDG standard was 10.9 μM and accordingly is even below the range (greater effectiveness) of the QC inhibitors known from U.S. Pat. No. 7,304,086 B2 with IC₅₀ values of 0.22 μM-14 μM.

Since the SQDG standard as reference substance for the compounds according to formula (I) inhibits QC, it is accordingly clearly demonstrated that compounds according to formula (I) are QC inhibitors.

1. Use of a compound of formula (I): 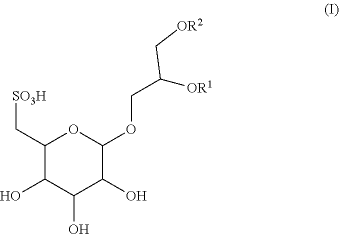
or a salt thereof, as glutaminyl cyclase (QC) inhibitor, wherein
R¹ is a hydrogen atom, —C(═O)C₁-C₂₆alkyl or —C(═O)C₂-C₂₆alkenyl; and
R² is a hydrogen atom, —C(═O)C₁-C₂₆alkyl or —C(═O)C₂-C₂₆alkenyl.

2. The use of the compound of formula (I) or a salt thereof according to claim 1, wherein
R¹ is a hydrogen atom, —C(═O)C₁₃-C₂₀alkyl or —C(=O)C₁₃-C₂₁alkenyl; and
R² is a hydrogen atom, —C(=O)C₁₃-C₂₁alkyl or —C(=O)C₁₃-C₂₁alkenyl.

3. The use of the compound of formula (I) or a salt thereof according to claim 1, wherein
R¹ is a hydrogen atom, a palmitoyl, palmitoleoyl, oleoyl, linoleoyl, linolenoyl, eicosatetraenoyl or an eicosapentaenoyl group; and
R² is a hydrogen atom, a palmitoyl, palmitoleoyl, oleoyl, linoleoyl, linolenoyl, eicosatetraenoyl or an eicosapentaenoyl group.

4. The use of the compound of formula (I) or a salt thereof according to claim 1, wherein the compound is selected from the group: 1,2-di-O-palmitoyl-3-O-(6′-deoxy-6′-sulfo-D-glycopyranosyl, 1-O-palmitoyl-2-O-linolenyl-3-O-(6′-deoxy-6′-sulfo-D-glycopyranosyl, 1-O-linolyl-2-O-palmitoyl-3-O-(6′-deoxy-6′-sulfo-D-glycopyranosyl, 1-O-palmitoyl-3-O-(6′-sulfo-α-quinovopyranosyl)-glycerol and 1-O-(6-deoxy-6-sulfoglucopyranosyl)-glycerol.

5. Use of a composition, comprising a compound of formula (I): 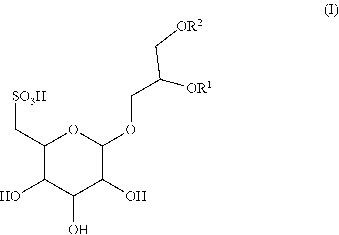
or a salt thereof, as QC inhibitor, wherein
R¹ is a hydrogen atom, —C(═O)C₁-C₂₆alkyl or —C(═O)C₂-C₂₆alkenyl; and
R² is a hydrogen atom, —C(═O)C₁-C₂₆alkyl or —C(═O)C₂-C₂₆-alkenyl.

6. The use of the composition according to claim 5, wherein the composition is an extract from a microalga.

7. The use of the composition according to claim 5, wherein the composition also contains a solvent.

8. The use of the composition according to claim 7, wherein the solvent is selected from: methanol, ethanol, isopropanol, water, chloroform, ethyl acetate, dichloromethane, acetone, diethyl ether, hexane and mixtures thereof.

9. The use of the composition according to claim 5, wherein the composition comprises two, three or more than three compounds according to formula (I) or salts thereof.

10. The use of the composition according to claim 5, wherein the composition comprises a further therapeutic agent selected from the group comprising acetylcholinesterase inhibitors, NMDA-receptor antagonists, neuroprotectors, anti-Parkinson medicaments, antidepressives, anxiolytic medicaments, antipsychotic medicaments, medicaments against multiple sclerosis, ACE inhibitors, diuretics, β-receptor blockers, nitrates, cardiac glycosides, calcium antagonists, lipid reducers, aggregation inhibitors, antihypoxaemics, coagulation inhibitors, cytostatics and antibiotics.

11. Method of preparing a compound of formula (I) described in claim 1, or a salt thereof, comprising:
(a) culturing at least one microalga;
(b) obtaining the algal biomass and disrupting the algal cells;
(c) extracting the disrupted algal cells from step (b) with methanol, comprising suspending the disrupted algal cells from step (b) in methanol, stirring the suspension, and separating the liquid methanol extract phase from the cell mass;
(d1) isolating a compound of formula (I), or a salt thereof, from the extraction product obtained in step (c).

12. Method of preparing an extract from a microalga, which extract contains at least one compound of formula (I) described in claim 1, or a salt thereof, wherein the method comprises:
(a) culturing the microalga;
(b) obtaining the algal biomass and disrupting the algal cells;
(c) extracting the disrupted algal cells from step (b) with methanol, comprising suspending the disrupted algal cells from step (b) in methanol, stirring the suspension, and separating the liquid methanol extract phase from the cell mass;
(d2) removing chlorophyll from the extraction product obtained in step (c).

13. A method of treating a subject, comprising administering to the subject an effective amount of a compound of formula (I): 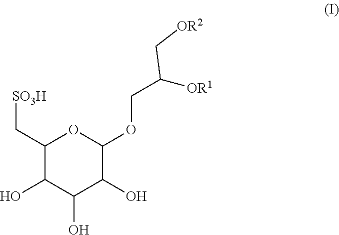
or a salt thereof, as glutaminyl cyclase (QC) inhibitor, wherein
R¹ is a hydrogen atom, —C(═O)C₁-C₂₆alkyl or —C(═O)C₂-C₂₆alkenyl; and
R² is a hydrogen atom, —C(═O)C₁-C₂₆alkyl or —C(═O)C₂-C₂₆alkenyl.

14. The method of claim 13 wherein
R¹ is a hydrogen atom, —C(═O)C₁₃-C₂₀alkyl or —C(=O)C₁₃-C₂₁alkenyl; and
R² is a hydrogen atom, —C(=O)C₁₃-C₂₁alkyl or —C(=O)C₁₃-C₂₁alkenyl.

15. The method of claim 13 wherein
R¹ is a hydrogen atom, a palmitoyl, palmitoleoyl, oleoyl, linoleoyl, linolenoyl, eicosatetraenoyl or an eicosapentaenoyl group; and
R² is a hydrogen atom, a palmitoyl, palmitoleoyl, oleoyl, linoleoyl, linolenoyl, eicosatetraenoyl or an eicosapentaenoyl group.

16. The method of claim 13 wherein the compound is selected from the group: 1,2-di-O-palmitoyl-3-O-(6′-deoxy-6′-sulfo-D-glycopyranosyl, deoxy-6′-sulfo-D-glycopyranosyl, 1-O-linolyl-2-O-palmitoyl-3-O-(6′-deoxy-6 sulfo-D-glycopyranosyl, 1-O-palmitoyl-3-O-(6′-sulfo-α-quinovopyranosyl)-glycerol and 1-O-(6-deoxy-6-sulfoglucopyranosyl)-glycerol.

17. The method of claim 13 wherein the subject is suffering from a QC-associated illness.

18. The method of claim 17 wherein administering the compound of formula (I) treats the QC-associated illness.

19. The method of claim 13 wherein a further therapeutic agent is administered to the subject selected from the group comprising acetylcholinesterase inhibitors, NMDA-receptor antagonists, neuroprotectors, anti-Parkinson medicaments, antidepressives, anxiolytic medicaments, antipsychotic medicaments, medicaments against multiple sclerosis, ACE inhibitors, diuretics, β-receptor blockers, nitrates, cardiac glycosides, calcium antagonists, lipid reducers, aggregation inhibitors, antihypoxaemics, coagulation inhibitors, cytostatics and antibiotics.

20. A pharmaceutical composition comprising a compound of claim 1.
